(19) United States
(12) Patent Application Publication
(10) Pub. No.: US 2019/0282525 A1
(43) Pub. Date: Sep. 19, 2019

(54) TIOPRONIN ORAL COMPOSITION

(71) Applicant: CRONUS RESEARCH LABS PRIVATE LIMITED, Hyderabad (IN)

(72) Inventor: Satya Srinivas Chetlapalli, Bangalore (IN)

(73) Assignee: CRONUS RESEARCH LABS PRIVATE LIMITED, Hyderabad (IN)

(21) Appl. No.: 16/356,990

(22) Filed: Mar. 18, 2019

(30) Foreign Application Priority Data
Mar. 19, 2018 (IN) 201841010051

(51) Int. Cl. A61K 31/197; A61P 13/12; A61K 9/20; A61K 9/28

(52) U.S. Cl. CPC A61K 31/197; A61P 13/12; A61K 9/2018; A61K 9/2054; A61K 9/2027; A61K 9/2063; A61K 9/2009; A61K 9/2031; A61K 9/2826; A61K 9/2059

(57) ABSTRACT

The invention relates to an oral tiopronin composition for reducing the dosing frequency in treatment of cysteinuria comprising of therapeutically effective amount of tiopronin and one or more pharmaceutically acceptable excipients.

### TECHNICAL FIELD OF THE INVENTION

The invention in general relates to pharmaceutical dosage forms. Particularly, the invention relates to oral composition of triopronin and the method for preparing the same.

### BACKGROUND ART

Cystinuria, a rare genetic condition, is caused by mutations in the genes that encode the two subunits of the amino acid transport, resulting in failure of absorption of filtered dibasic amino acids including cystine in the proximal tubules. The very limited solubility of cystine in the physiological range of urine pH, leads to complicated and recurrent kidney stone formation leading to a higher risk of chronic kidney disease.

Sulfhydroxyl compounds which act as thiol-binding agents, such as D-penicillamine or tiopronin (α-mercaptopropionylglycine), are reported to reduce the formation of cystine crystals by reacting with cystine and generating more soluble mixed disulfide compounds (drug-cysteine complex).

Presently available dosage form for Tiopronin in the market is Thiola. THIOLA is indicated for the prevention of cystine (kidney) stone formation in patients with severe homozygous cystinuria with urinary cystine greater than 500 mg/day. The goal of therapy is to reduce urinary cystine concentration below its solubility limit.

The decrement in urinary cystine produced by tiopronin is generally proportional to the dose. A reduction in urinary cystine of 250-350 mg/day at a tiopronin dosage of 1 g/day, and a decline of approximately 500 mg/day at a dosage of 2 g/day, might be expected.

Tiopronin have significant side effects within the usual range of therapeutic dosage (1000-2000 mg/day), including foul odor, nausea, fever, fatigue, skin rash, premature skin aging, proteinuria and hypersensitivity, which restricts their use to patients who are unable to control stone formation with high fluid intake, dietary modification and urine alkalinisation.

The marketed tiopronin dosage form is available in 100 mg strength only, and a patient has to take 20 tablets per day to meet dose of 2000 mg/day to reduce the urinary cysteine of 500 mg/day. Daily intake of 20 tablets leads to poor patient compliance and also increased side effects which also is another reason for poor patient compliance.

### SUMMARY OF THE INVENTION

Herein disclosed is tiopronin oral composition for reducing the dosing frequency and thereby improving the treatment compliance by the patients. The oral tiopronin composition for reducing the dosing frequency in treatment of cysteinuria comprises of therapeutically effective amount of tiopronin and one or more pharmaceutically acceptable excipients. The oral tiopronin composition as disclosed herein comprises of 200 mg of tiopronin, one or more fillers, one or more disintegrants/disintegrating agents, one or more binders, one or more glidants/gliding agents, one or more lubricants lubricating agents or a combination thereof. The oral tiopronin composition is a tablet and preferably a sugar coated tablet.

The filler is selected from the group consisting of directly compressible starches, hydrolysed starches, Lactose and its derivates dextrose, sorbitol, microcrystalline cellulose, dibasic calcium phosphate dehydrate, Calcium sulphate dehydrate or a combination thereof.

The disintegrant/disintegrating agent is selected from the group consisting of starches, clays, cellulose and its derivates including hydroxypropyl cellulose, cross linked polymers, modified starches including sodium starch glycolate, magnesium aluminium silicate, crosscarmalose, cross povidone, strach derivatives, alginates, Polyvinylpyrrolidone or a combination thereof.

The binder is selected from the group consisting of acacia, gelatin, starch, pregelatinized starch, polyvinylpyrrolidone, glucose, povidone, cellulose derivatives including methyl cellulose, ethyl cellulose, carboxymethyl cellulose, microcrystallince cellulose, hydroxyethyl cellulose hydroxypropylmethyl cellulose (HPMC), tragacanth, alginate derivatives inclusing sodium alginate, synthetic polymers, shellac or a combination thereof.

The glidant/gliding agent is selected from the group consisting of talc, colloidal silicone dioxide, asbestos free starch, corn starch, maize starch, calcium phosphate, calcium silicate, powdered cellulose, magnesium trisilicate, silicon dioxide, silica derivates including colloidal silica, colloidal silica anhydrous or a combination thereof.

The lubricant/lubricating agent is selected from the group consisting of talc, stearic acid, magnesium stearate, calcium stearate, waxes, polyethylene glycol, surfactants or vegetable oil.

### DETAILED DESCRIPTION OF THE INVENTION

The invention disclosed herein provides a tiopronin oral composition for reducing the dosing frequency and thereby improving the treatment compliance by the patients. The oral tiopronin composition for reducing the dosing frequency in treatment of cysteinuria comprises of therapeutically effective amount of tiopronin and one or more pharmaceutically acceptable excipients. The oral tiopronin composition as disclosed herein comprises of 200 mg of tiopronin or its pharmaceutically acceptable salts.

According to one embodiment the triopronin is tiopronin hydrochloride. The oral tiopronin composition comprises one or more fillers, one or more disintegrants/disintegrating agents, one or more binders, one or more glidants/gliding agents, one or more lubricants lubricating agents or a combination thereof. According to one embodiment the oral tiopronin composition is a sugar coated tablet.

The filler is selected from the group consisting of directly compressible starches, hydrolysed starches, Lactose and its derivates dextrose, sorbitol, microcrystalline cellulose, dibasic calcium phosphate dehydrate, Calcium sulphate dehydrate or a combination thereof.

The disintegrant/disintegrating agent is selected from the group consisting of starches, clays, cellulose and its derivates including hydroxypropyl cellulose, cross linked polymers, modified starches including sodium starch glycolate, magnesium aluminium silicate, crosscarmalose, cross povidone, strach derivatives, alginates, Polyvinylpyrrolidone or a combination thereof.

The binder is selected from the group consisting of acacia, gelatin, starch, pregelatinized starch, polyvinylpyrrolidone, glucose, povidone, cellulose derivatives including methyl cellulose, ethyl cellulose, carboxymethyl cellulose, microcrystallince cellulose, hydroxyethyl cellulose hydroxypropylmethyl cellulose (HPMC), tragacanth, alginate derivatives inclusing sodium alginate, synthetic polymers, shellac or a combination thereof.

The glidant/gliding agent is selected from the group consisting of talc, colloidal silicone dioxide, asbestos free starch, corn starch, maize starch, calcium phosphate, calcium silicate, powdered cellulose, magnesium trisilicate, silicon dioxide, silica derivates including colloidal silica, colloidal silica anhydrous or a combination thereof.

The lubricant/lubricating agent is selected from the group consisting of talc, stearic acid, magnesium stearate, calcium stearate, waxes, polyethylene glycol, surfactants or vegetable oil.

### Example 1—Compositions of Core Tablet

| | | Amount (mg/Unit) | | | |
|---|---|---|---|---|---|
| S. No. | Name of the Active/Excipient | F1 | F2 | F3 | F4 |
| 1 | Tiopronin | 200 | 200 | 200 | 200 |
| 2 | Lactose anhydrous | 40 | 40 | 40 | 40 |
| 3 | L-Hydroxypropylcellulose | 20 | 20 | 20 | 20 |
| 4 | Polyvinylpyrrolidone | 4 | 4 | 4 | 4 |
| 5 | Isopropyl alcohol | q.s. | q.s. | | |
| 6 | Ethyl cellulose | 13 | 13 | 19.50 | 10.25 |
| 7 | L-Hydroxypropyl cellulose | 20 | 20 | 13 | 6.66 |
| 8 | Talc | 20 | 20 | 13 | 6.66 |
| 9 | Lactose anhydrous | 60 | | 30.70 | 30.77 |
| 10 | Magnesium stearate | 13 | 13 | 20 | 10.26 |
| | Core tablet's weight | 390 | 330 | 390 | 390 |

Add Polyvinylpyrrolidone to isopropyl alcohol and stir the solution until clear solution observed without any lumps. Tiopronin, Lactose anhydrous Part-1 & L-Hydroxypropylcellulose (LH 21) into RMG and mixed well for 10 minutes with impeller at fast speed. Slowly add Polyvinylpyrrolidone solution into rapid mixer granulator over period of 60-90 seconds at impeller slow speed and rake the material. Knead the wet mass for 30-60 seconds at impeller fast speed and chopper fast speed until get a good granular mass. Unload the wet granular mass into FBD bowl.

Air-dry the wet granules for 5 minutes to ensure proper fluidization. Dried the wet granular mass in fluid bed drier at an inlet temperature of 55° C.±5° C. & outlet temperature 45±5° C. until get moisture content of NMT 1.5% L achieved.

Milled the dried granules and sift through 20 ASTM sieve (850μ). Add the ethyl cellulose, Hydroxypropylcellulose (Part 2), Lactose anhydrous (Part 2) & Talc and blend it for 10 minutes. Finally add magnesium stearate and blend it for 5 min. Compressed the lubricated blend using the BB type, 10.00 mm, Round shape, Deep concave plain punches.

### Example 2—Coating

| | Coating Excipients | | | |
|---|---|---|---|---|
| S. No. | (7.45% w/w) | F5 | F6 | F7 |
| 1 | Hydroxypropyl cellulose | 5.67 | 4.8 | 0 |
| 2 | Eudragit E1 00 | 21.27 | 18 | 10 |
| 3 | Triethyl citrate | 2.13 | 1.8 | 1 |
| 4 | Isopropyl alcohol | q.s. | q.s. | q.s. |
| 5 | Acetone | q.s. | q.s. | q.s. |

Add Eudragit E 100 to the Acetone & IPA mixture under continuous stirring and stir the solution for 1 hr. After dissolving the Eudragit E-100 add Hydroxyl propyl cellulose slowly under continuous stirring and stir the solution for 30-45 minutes. Add triethyl citrate to above solution and stir the solution for 10-15 minutes and filtered the dispersion through #200 (750 nylon cloth. The coated tablets are dried for 120 minutes at 40° C. bed temperature.

### Example 3—Sub Coating

| | Coating Excipients | | | |
|---|---|---|---|---|
| S. No. | (7.45% w/w) | F5 | F6 | F7 |
| 1 | Sucrose | 118.18 | 100 | 80 |
| 2 | Calcium carbonate | 40.99 | 34.68 | 50 |
| 3 | Talc | 9.60 | 8.12 | 4 |
| 4 | Purified water | q.s. | q.s. | q.s. |

Heat purified water in steam-jacketed vessel to 80-90° C. Add sucrose to heat water under and Stir the solution without any lumps for 20-30 minutes. After dissolving the sucrose add Calcium Carbonate & Talc and homogenize the solution using homogenizer/colloidal mill for 15-30 minutes. Filtered the coating solution with #100 (1500 nylon cloth in to a clean SS vessel.

Load Eudragit coated tablets into coating pan and perform the coating using the below mentioned parameters with intermittent spray pattern. After coating tablets to be dried for 30 minutes at 45° C. bed temperature.

### Example 4—Syrup Coating

| S. No. | Name of the material | F11 | F12 | F13 |
|---|---|---|---|---|
| 1 | Sucrose | 47.27 | 40.00 | 38.00 |
| 2 | Talc | 10.05 | 3.40 | 15.00 |
| 3 | Titanium dioxide | 4.02 | 8.00 | 14.00 |
| 4 | Water | q.s. | q.s. | q.s. |

Heat purified water in steam-jacketed vessel to 80-90° C. Add sucrose to heat water and stir the solution without any lumps for 20-30 minutes. After dissolving sucrose add Talc & Titanium dioxide and homogenize the solution using homogenizer/colloidal mill for 15-30 minutes. Filter the suspension with #100 (150μ) nylon cloth in to a clean SS vessel. Load the sub coated tablets into coating pan and perform the coating using the below mentioned parameters. Spray, pause followed by dry condition has followed during syrup coating. After coating dried the tablets for 30 minutes at 40° C. bed temperature.

### Example 5—Polishing

| S. No. | Excipients | F14 | F15 | F16 |
|---|---|---|---|---|
| 1 | Opagloss 6000P | 0.82 | 1.00 | 0.60 |
| 2 | Isopropyl alcohol | q.s. | q.s. | q.s. |

Dispense required quantity of Isopropyl alcohol required to make 5% w/w solution of polishing coating material. Add Opagloss 6000P to the isopropyl alcohol under stirring and continue the stirring for 10-15 minutes until the homogeneous solution observed. Load the syrup coated tablets into pan and perform the process as per given below parameters. After coating tablets are dried for 30 minutes at 40° C. bed temperature.

We claim:

1. An oral tiopronin composition for reducing the dosing frequency in treatment of cysteinuria comprising of therapeutically effective amount of tiopronin and one or more pharmaceutically acceptable excipients, wherein the amount of tiopronin is 200 mg.

2. The oral tiopronin composition of claim 1 wherein the pharmaceutically acceptable excipient is selected from the group comprising of fillers, disintegrants, binders, glidants, lubricants or a combination thereof.

3. The oral tiopronin composition of claim 1 wherein the filler is selected from the group consisting of directly compressible starches, hydrolysed starches, Lactose and its derivates dextrose, sorbitol, microcrystalline cellulose, dibasic calcium phosphate dehydrate, Calcium sulphate dehydrate or a combination thereof.

4. The oral tiopronin composition of claim 1 wherein the disintegrant is selected from the group consisting of starches, clays, cellulose and its derivates including hydroxypropyl cellulose, cross linked polymers, modified starches including sodium starch glycolate, magnesium aluminium silicate, crosscarmalose, cross povidone, strach derivatives, alginates, Polyvinylpyrrolidone or a combination thereof.

5. The oral tiopronin composition of claim 1 wherein the binder is selected from the group consisting of acacia, gelatin, starch, pregelatinized starch, polyvinylpyrrolidone, glucose, povidone, cellulose derivatives including methyl cellulose, ethyl cellulose, carboxymethyl cellulose, microcrystallince cellulose, hydroxyethyl cellulose hydroxypropylmethyl cellulose (HPMC), tragacanth, alginate derivatives inclusing sodium alginate, synthetic polymers, shellac or a combination thereof.

6. The oral tiopronin composition of claim 1 wherein the glidant is selected from the group consisting of talc, colloidal silicone dioxide, asbestos free starch, corn starch, maize starch, calcium phosphate, calcium silicate, powdered cellulose, magnesium trisilicate, silicon dioxide, silica derivates including colloidal silica, colloidal silica anhydrous or a combination thereof.

7. The oral tiopronin composition of claim 1 wherein the lubricant is selected from the group consisting of talc, stearic acid, magnesium stearate, calcium stearate, waxes, polyethylene glycol, surfactants or vegetable oil.

8. The oral tiopronin composition of claim 1 is a tablet

9. The oral tiopronin composition of claim 8 wherein the tablet is a sugar coated tablet.
